(19) United States
(12) Patent Application Publication
(10) Pub. No.: US 2012/0253204 A1
(43) Pub. Date: Oct. 4, 2012

(54) SYSTEMS AND METHODS FOR PHOTOTHERAPEUTIC TREATMENT OF RECTAL DISEASES

(75) Inventor: Sharon BEN-YEHUDA, Rechovot (IL)

(73) Assignee: Photopill Medical Ltd., Yokneam (IL)

(21) Appl. No.: 13/494,199

(22) Filed: Jun. 12, 2012

Related U.S. Application Data
(63) Continuation-in-part of application No. PCT/IL2012/050073, filed on Mar. 7, 2012.
Application No. PCT/IL2012/050073 is a continuation-in-part of application No. PCT/IL2011/000257, filed on Mar. 17, 2011.
(60) Provisional application No. 61/496,012, filed on Jun. 12, 2011.
(60) Provisional application No. 61/537,095, filed on Sep. 21, 2011.
(60) Provisional application No. 61/314,762, filed on Mar. 17, 2010.

(51) Int. Cl. A61N 5/06; A61B 6/00

(52) U.S. Cl. 600/476; 607/92

(57) ABSTRACT

A phototherapeutic system for providing phototherapy to a patient's lower parts of a gastrointestinal (GI) tract, the phototherapeutic system including a capsule sized and shaped for insertion into the rectum, the capsule including one or more phototherapeutic light sources. A method for intra-luminal phototherapy of the lower parts of the gastrointestinal (GI) tract including inserting a phototherapeutic device including one or more phototherapeutic light sources into the rectum, activating the light sources, and providing light at a dose suitable for providing a phototherapeutic effect. Related apparatus and methods are also described.

### RELATED APPLICATION/S

This application claims the benefit of priority under 35 USC 119(e) of U.S. Provisional Patent Application No. 61/496,012 filed Jun. 12, 2011.

This application is also a Continuation-in-Part (CIP) of PCT Patent Application No. PCT/IL2012/050073 having International filing date of Mar. 7, 2012, which claims the benefit of priority under 35 USC 119(e) of U.S. Provisional Patent Application No. 61/537,095 filed Sep. 21, 2011, and which is also a Continuation-in-Part (CIP) of PCT Patent Application No. PCT/IL2011/000257 having International filing date of Mar. 17, 2011, which claims the benefit of priority under 35 USC 119(e) of U.S. Provisional Patent Application No. 61/314,762 filed Mar. 17, 2010.

The contents of all the above applications are all incorporated herein by reference.

### FIELD AND BACKGROUND OF THE INVENTION

The present invention, in some embodiments thereof, relates to a phototherapy device which may be used for treating diseases of the gastrointestinal (GI) tract, and in particular, for treating lower parts of the GI tract, such as the descending colon, the sigmoid and the rectum, using phototherapy.

Light therapy, conventionally referred to as “phototherapy”, comprises exposing living tissue to light to treat a disease of the organism or tissue. The exposure is typically provided in accordance with a particular protocol tailored to the disease that defines spectrum and intensity of light used to illuminate the tissue and total energy deposited in the tissue by the light. The light may be generated using any of various suitable light sources, such as lasers, light emitting diodes (LEDs) and fluorescent lamps.

Phototherapy is generally applied to relatively easily accessible tissue regions, such as external regions of the skin and the mucosa lining the mouth or nose, and is used to treat acne, psoriases, eczema, vitiligo (in which damage to skin pigment cells results in white skin patches) and skin-based lymphoma, gingivitis, gum inflammations, oral ulcers, and allergic rhinitis.

A similar-sounding but different field from phototherapy is Photodynamic Therapy (PDT). Most modern PDT applications involve three key components: a photo-sensitizer, a light source and tissue oxygen. The wavelength of the light source needs to be appropriate for exciting the photo-sensitizer to produce reactive oxygen species. The combination of these three components leads to the chemical destruction of any tissues which have either selectively taken up the photo-sensitizer or have been locally exposed to light. In understanding the mechanism of PDT it is important to distinguish it from other light-based and laser therapies such as laser wound healing and rejuvenation which do not require a photo-sensitizer.

GI tract diseases, known as IBD (Inflammatory Bowel Diseases), are a group of bowel diseases, which include inflammations and ulcerations of the entire GI tract. Within the IBD family, the most known and common diseases are Crohn's disease and Ulcerative Colitis. These diseases are manifested throughout the Gastro Intestinal tract (from mouth to anus) and as such, can appear in the rectum as well.

In the case of Ulcerative Colitis or Crohn's disease that affects only the rectum, the disease may be related to as Crohn's Proctitis. or Ulcerative Proctitis.

Proctitis appears as an inflammation of the anus and the lining of the rectum, often affecting only the last 6 inches of the rectum.

Proctitis can have many possible causes. Proctitis may occur idiopathically, named idiopathic Proctitis. Other causes for Proctitis include damage by irradiation, for example in radiation therapy for cervical cancer and prostate cancer, or as a sexually transmitted infection, as in lymphogranuloma venereum and herpes proctitis. Proctitis is also linked to stress, and recent studies suggest a celiac disease-associated “Proctitis”, which can result from intolerance to gluten.

A common cause of Proctitis is engaging in anal sex with multiple partners infected with sexual transmitted diseases.

International Patent Application Publication WO 2011/114332, titled “Capsule Phototherapy”, discloses a swallowable capsule suitable for providing phototherapy to a region of a patient's gastrointestinal (GI) tract, the capsule comprising one or more light sources emitting in the visible and/or NIR ranges and optical elements for shaping the light beam produced by said light sources, such that said light source(s) and said optical elements are capable of delivering an effective therapeutic dose to a target site within the GI tract. The disclosure further provides a method for intraluminal phototherapy of the gastrointestinal tract using a swallowable capsule as described hereinabove.

International Patent Application Publication WO 2008/012701, titled “Capsule camera with variable illumination of the surrounding tissue”, discloses an ingestible capsule and method for in vivo imaging and/or treatment of one or more diseased areas of interest within the gastrointestinal tract of an animal or human being. The capsule comprises an image sensor; a lens system for focusing images onto the image sensor; at least one light source for illumination of the tissue area of interest, the at least one light source optionally being capable of providing optical therapeutic treatment to the diseased areas; a variable lens system located in front of the at least one light source, wherein the variable lens system comprises beam steering means and focusing means for directing and focusing the light beams from the at least one light source onto the diseased tissue areas,—a control unit in communication with the image sensor, the at least one light source, and variable lens system, a power source for powering the image sensor, the at least one light source and the control unit; and a non-digestible, transparent outer protective shell configured to pass through the gastrointestinal tract, housing within the image sensor, the lens system, the at least one light source, the variable lens system, the control unit and the power source.

An article entitled “Autonomous Device for Photostimulation of the Gastrointestinal Tract Immunity” by Sergey A. Naumov Vladimir N. Dyrin, Sergey M. Vovk, Evgeny Y. Petrov, Vladimir V. Udut and Elena V. Borodulina, published in Proc. SPIE 3907, 433 (2000); doi:10.1117/12.386284, describes a very small optoelectronic device emitting light in the red and green band has been developed as a small capsule consisting of two semispheres connected with light-transmitted coupling. The device—a phototablet permits to irradiate all parts of the gastro-intestinal tract (GIT) including the immunocompetent formations of the small intestine—Peyer's patches responsible for production of secretory immunoglobulins A (IgA). The main mechanisms of realizing endogenic phototherapy using a phototablet begin functioning when irradiating both the walls of the GIT organs and its contents. The results of clinical trials of the phototablet testify to a favorable effect of endogenic therapy on the human organism in asthenic syndrome, some types of deficiency in the immunity function, in dysbioses, the syndrome of large intestine irritation, duodenostasis, etc. After endogenic phototherapy the patients had an increased level of lysozyme, leukocytes, a number of lactobacteria. There were no side effects when using a phototablet. Indications and contraindications for endogenic phototherapy were represented. Thus, the method of endogenic phototherapy allows us to have an effective and direct influence on the immunocompetent cells of GIT organs without medicamental agents and antigens that makes it possible to use the phototablet in medicine on a large scale.

US published patent application 2009/0177033 of Hendriks at al., describes an application which relates to an ingestible capsule and method for in vivo imaging and/or treatment of one or more diseased areas of interest within the gastrointestinal tract of an animal or human being. The capsule comprises an image sensor; a lens system for focusing images onto the image sensor; at least one light source for illumination of the tissue area of interest, the at least one light source optionally being capable of providing optical therapeutic treatment to the diseased areas; a variable lens system located in front of the at least one light source, wherein the variable lens system comprises beam steering means and focusing means for directing and focusing the light beams from the at least one light source onto the diseased tissue areas, a control unit in communication with the image sensor, the at least one light source, and variable lens system, a power source for powering the image sensor, the at least one light source and the control unit; and a non-digestible, transparent outer protective shell configured to pass through the gastrointestinal tract, housing within the image sensor, the lens system, the at least one light source, the variable lens system, the control unit and the power source.

Additional background art includes:
an article titled: “Compartmental Transit and Dispersion Model Analysis of Small Intestine Transit Flow in Humans”, by Lawrence X. Yu, John R. Crison and Gordon L. Amidon, published in International Journal of Pharmaceutics, Vol 40; 1999;
an article titled: “Relationship of Gastric Emptying and Volume Changes After Solid Meal in Humans”, by Duane D. Burton, H. Jae Kim, Michael Camilleri, Debra A. Stephens, Brian P. Mullan, Michael K. O'Connor, and Nicholas J. Talley, published in Am J Physiol Gastrointest Liver Physiol 289, 2005;
an article by Wirtz et al., published in Nature Protocols, Vol. 2, pp. 541-546, 2007;
PCT published patent application WO 2009/102445 of Bandy et al;
US published patent application 2008/0106596 of Iddan et al;
US published patent application 2004/0249245 of Irion;
US published patent application 2004/0106849 of Cho et al;
US published patent application 2003/0214579 of Iddan; and
U.S. Pat. No. 5,464,436 to Smith.

The disclosures of all references mentioned above and throughout the present specification, as well as the disclosures of all references mentioned in those references, are hereby incorporated herein by reference.

### SUMMARY OF THE INVENTION

The present invention, in some embodiments thereof, relates to a phototherapy device which may be used for treating diseases of the gastrointestinal (GI) tract, and in particular, for treating the Descending Colon, Sigmoid and Rectum using phototherapy.

Phototherapy has proven to be beneficial for IBD, and can be used to improve or treat rectum diseases which involve ulcerations and/or inflammations, such as Crohn's disease, Ulcerative Colitis, Diverticulitis or Proctitis.

In above-mentioned PCT Patent Application number PCT IL2011/000257 a capsule for providing phototherapy for the GI tract is described. Such a capsule can travel through the GI tract from mouth to anus, administering a measured dose of phototherapeutic illumination to the GI tract.

In some embodiments, a phototherapy emitting device, such as a capsule or an illuminating rectal tube, is applied like a suppository into the rectum, and provides phototherapy to the rectum walls.

The term “capsule” will be used throughout the present specification and claims to stand for a capsule, and/or an illuminating rectal tube, and/or a capsule connected to an applicator and/or handle. Using a single term is not meant to limit the teachings and claims of the invention to a capsule only, and persons skilled in the art will know how to apply the teaching of the capsule to an embodiment of an illuminating rectal tube.

When it is desired to provide phototherapy to the rectum or colon, some embodiments of the present invention take advantage of the fact that the rectum, sigmoid and descending colon together are shorter than an entire GI tract, and more accessible. More configurations of a phototherapeutic device are provided than are possible for a device which negotiates passage of the entire GI tract.

According to an aspect of some embodiments of the present invention there is provided a phototherapeutic system for providing phototherapy to a patient's lower parts of a gastrointestinal (GI) tract, the phototherapeutic system including a capsule sized and shaped for insertion into the rectum, the capsule including one or more phototherapeutic light sources.

According to some embodiments of the invention, the capsule is longer than 4 centimeters. According to some embodiments of the invention, the capsule diameter is more than 2 centimeters.

According to some embodiments of the invention, further including a centering component configured to center the capsule relative to a diameter of the rectum. According to some embodiments of the invention, the centering component is configured to exert pressure on walls of the rectum, thereby flattening folds in the walls. According to some embodiments of the invention, the centering component is an inflatable balloon surrounding the capsule. According to some embodiments of the invention, the centering component is substantially transparent to a wavelength used for the providing phototherapy.

According to some embodiments of the invention, the phototherapeutic light sources includes a plurality of phototherapeutic light sources, and the phototherapeutic light sources are distributed along a length of the capsule.

According to some embodiments of the invention, the phototherapeutic light sources include a plurality of phototherapeutic light sources, the phototherapeutic light sources are distributed along a length of the capsule, and the centering component surrounds the phototherapeutic light sources distributed along the length of the capsule.

According to some embodiments of the invention, the capsule further includes a movement determination unit for calculating speed of movement of the capsule in the rectum.

According to some embodiments of the invention, the capsule further includes a controller unit for activating one or more of the one or more light sources for delivering a therapeutic illumination dose to a target site in the GI tract, based, at least in part, on a determined speed calculated based, at least in part, on movement determined by the movement determination unit.

According to some embodiments of the invention, further including a controller unit, external to the capsule, for activating one or more of the one or more light sources for delivering a therapeutic illumination dose to a target site in the GI tract, based, at least in part, on a determined speed calculated based, at least in part, on movement determined by the movement determination unit.

According to some embodiments of the invention, the controller unit is included in an applicator connected to the capsule. According to some embodiments of the invention, the controller unit is included in a computer configured to be external to a patient's body.

According to some embodiments of the invention, further including a depth-of-insertion measurement component. According to some embodiments of the invention, the depth-of-insertion measurement component is an electronic depth-of-insertion measurement component connected to the controller unit.

According to some embodiments of the invention, the controller unit is configured to indicate to a physician when a therapeutic dose has been completely administered to a location in the rectum, based, at least in part, on the determined movement and on a required dose. According to some embodiments of the invention, the controller unit is configured to indicate to a physician an indication for changing a speed of withdrawal of the capsule from a patient's rectum based, at least in part, on the determined movement and on a required dose.

According to some embodiments of the invention, further including an applicator for inserting the capsule into the rectum, wherein the applicator is shaped for manipulating from outside a patient's body. According to some embodiments of the invention, the applicator includes a mechanical connector for connecting to the capsule, and wherein the applicator is configured to enable disconnection from the capsule when the capsule is within a patient's rectum.

According to some embodiments of the invention, further including a power supply built within the capsule. According to some embodiments of the invention, further including a power cable configured for connecting the capsule to a power supply outside a patient's body.

According to an aspect of some embodiments of the present invention there is provided a method for intra-luminal phototherapy of the lower parts of the gastrointestinal (GI) tract including inserting a phototherapeutic device including one or more phototherapeutic light sources into the rectum, activating the light sources, and providing light at a dose suitable for providing a phototherapeutic effect.

According to some embodiments of the invention, further including substantially centering the phototherapeutic device relative to a circumference of the rectum. According to some embodiments of the invention, the centering exerts pressure on walls of the rectum, thereby flattening folds in the walls. According to some embodiments of the invention, the centering includes inflating a balloon surrounding the phototherapeutic device.

According to some embodiments of the invention, further including measuring a depth-of-insertion of the phototherapeutic device in the rectum by an electronic depth-of-insertion measurement component, and providing the light based, at least in part, on the measuring.

According to some embodiments of the invention, the phototherapeutic device includes a capsule connected to an applicator, and further including detaching the applicator and removing the applicator from the rectum, leaving the capsule inside the rectum.

According to some embodiments of the invention, further including measuring movement of the phototherapeutic device relative to the rectum, and using the movement to calculate the dose of therapeutic lighting received by walls of the rectum, wherein the measurement is used to indicate to a user at what rate the phototherapeutic device should be pulled out of the rectum while therapeutic light is being provided.

According to some embodiments of the invention, the phototherapeutic device further includes a camera, and the method for intra-luminal phototherapy further includes using the camera to locate a diseased area of the rectum. According to some embodiments of the invention, further including recording images of the diseased area.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE DRAWINGS

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIG. 1 is a simplified drawing of a human GI tract;

FIG. 2 is a simplified drawing of a capsule for providing phototherapeutic illumination according to an example embodiment of the invention;

FIG. 3 is a simplified drawing of a capsule in place within a rectum of a patient, according to an example embodiment of the invention;

FIG. 4 is a simplified drawing of a bottom of a capsule and of an applicator for inserting the capsule into the rectum, according to an example embodiment of the invention;

FIGS. 5A, 5B and 5C are more detailed simplified illustrations of technical details of the applicator according to an example embodiment of the invention;

FIG. 6A is a simplified illustration of a therapeutic illumination tube constructed according to an example embodiment of the invention, inserted into a rectum;

FIG. 6B is a simplified illustration of a therapeutic illumination tube constructed according to an example embodiment of the invention;

FIG. 6C is a simplified illustration of a therapeutic illumination tube having an electro-optical depth measurement component constructed according to an example embodiment of the invention;

FIG. 6D is a simplified graphic illustration of reflection intensity from several light detectors along the therapeutic illumination tube with the electro-optical depth measurement component of FIG. 6C;

FIG. 7A is a simplified illustration of a therapeutic illumination tube with an inflatable balloon according to an example embodiment of the invention;

FIG. 7B is a simplified illustration of a therapeutic illumination tube with an inflatable balloon according to another example embodiment of the invention;

FIG. 7C is a simplified illustration of a therapeutic illumination tube with a camera according to an example embodiment of the invention;

FIG. 8 is a simplified illustration of a therapeutic illumination tube with an inflatable balloon inserted into a rectum according to an example embodiment of the invention;

FIG. 9 is a simplified flow chart illustration of a method for providing phototherapy to the rectum according to an example embodiment of the invention; and

FIG. 10 is a simplified flow chart illustration of a method for providing phototherapy to the rectum according to an example embodiment of the invention.

### DESCRIPTION OF SPECIFIC EMBODIMENTS OF THE INVENTION

The present invention, in some embodiments thereof, relates to a phototherapy device which may be used for treating diseases of the lower gastrointestinal tract, and in particular, for treating the rectum, using phototherapy.

The term “rectum” will be used throughout the present specification and claims to stand for the rectum and/or the sigmoid and/or the descending colon. Using a single term is not meant to limit the teachings and claims of the invention to the rectum only, only for convenience.

In some embodiments, a phototherapeutic device is placed into the rectum and/or colon via the anus, similarly to putting other devices, such as a colonoscope, or suppositories, into the rectum and/or colon.

Whereas the capsule of above-mentioned PCT Patent Application No. PCT IL2011/000257 is un-tethered, embodiments of the phototherapeutic device described herein may be similarly un-tethered, or may be tethered (for example with a cable leading to a power supply external to the body), or may be connect to an applicator shaped for inserting the phototherapeutic device into the rectum.

In some embodiments, a power supply of the phototherapeutic device may be contained within the device, similarly to the phototherapy capsule of the above-mentioned PCT Patent Application number PCT IL2011/000257. However, in some embodiments the power supply may be contained in the above-mentioned applicator, and in some embodiments the power supply may be external to the patient's body, connected by a power supplying cable to the phototherapeutic device within the rectum.

The above-mentioned PCT Patent Application No. PCT IL2011/000257 teaches a movement measurement component of the phototherapy capsule, for controlling the phototherapeutic dose provided. In some embodiments, the phototherapeutic device includes a speed measurement component, based on the movement, which measures speed of movement of the phototherapeutic device relative to the rectum walls, and controls the phototherapeutic dose provided.

In some embodiments the phototherapeutic device includes a timer, and provides an indication to a physician when a correct dose has been applied to the rectal wall. As a result of the indication, the physician may optionally turn off the phototherapeutic illumination by an external switch or by controlling the power supply.

In some embodiments, a correct dose is being applied when the phototherapeutic device moves along the rectum at a rate of 30 seconds per centimeter, or 2 minutes per centimeter, and up to 6 minutes per centimeter.

In some embodiments the phototherapeutic device includes the speed measurement component, and provides an indication to a physician when a correct dose has been applied to the rectal wall. As a result of the indication, the physician may optionally turn off the phototherapeutic illumination by an external switch or by controlling the power supply.

In some embodiments the phototherapeutic device includes the speed measurement component, and provides an indication to a physician at what speed to withdraw the phototherapeutic device so that a correct dose is applied to the rectal wall. As a result of the indication, the physician may optionally pull a tethered device, or pull the applicator. The speed indication may optionally be provided as a form of a two or three indicators communicating “faster”, “slower”, or “just right” to a physician.

In some embodiments, the phototherapeutic device includes a component for centering the illumination source in the rectum, so that the rectal walls around the illumination source are equi-distant from the source, receiving equal illumination intensity.

In some embodiments, the phototherapeutic device includes a component for stretching the rectal walls, so that the rectal walls around the illumination source are flattened, eliminating potential folds which might not receive illumination.

A typical diameter of the small intestine can be 2-3 centimeters, whilst a typical diameter of the large intestine can be 5-7 centimeters.

In some embodiments of the invention, the capsule is made long enough so as not to flip over in the rectum. In some embodiments of the invention, the capsule is longer than 4, 5, 6, 7, 10, or even 15 centimeters. Such a length may be too long, and undesirable, for a capsule which needs to travel along an entire length of a GI tract, from mouth to rectum.

In some embodiments of the invention, the capsule is sized and shaped so as to be able to penetrate to a depth of up to 15-25 centimeters from the anus, and no more. That enables the capsule to be thicker than a capsule which would be desirable for a capsule which needs to travel along an entire length of a GI tract, from mouth to rectum. In some embodiments, the diameter of the capsule is larger than 2 or 3 centimeters, which may be undesirable if the capsule is to be swallowed and travel through the entire GI tract, but still be acceptable when introduced to a body through the anus and into the descending colon, and/or the sigmoid, and/or the rectum.

Before explaining at least one embodiment of the invention in detail, it is to be understood that the invention is not necessarily limited in its application to the details of construction and the arrangement of the components and/or methods set forth in the following description and/or illustrated in the drawings and/or the Examples. The invention is capable of other embodiments or of being practiced or carried out in various ways.

The Colon and the Rectum

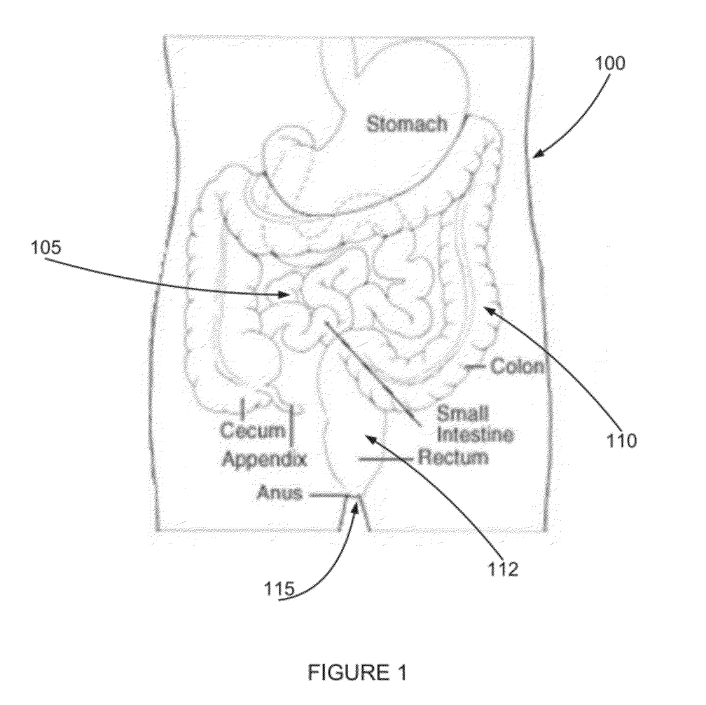

Reference is now made to FIG. 1, which is a simplified drawing of a human GI tract 105. FIG. 1 depicts a portion of a human body approximating a torso 100, within which the GI tract 105 is depicted. Two regions of the GI tract are specifically pointed out—the colon 110 and the rectum 110. The anus 115 is also pointed out, being an end of, and an exit from, the GI tract.

It is noted that the colon 110 and the rectum 112 have a greater diameter than some other parts of the GI tract. A phototherapeutic device which can be inserted via the anus 115 into the rectum 112 and/or the colon 110 can be larger than a phototherapeutic capsule as described in above-mentioned PCT Patent Application number PCT IL2011/000257.

An Example Embodiment: a Capsule

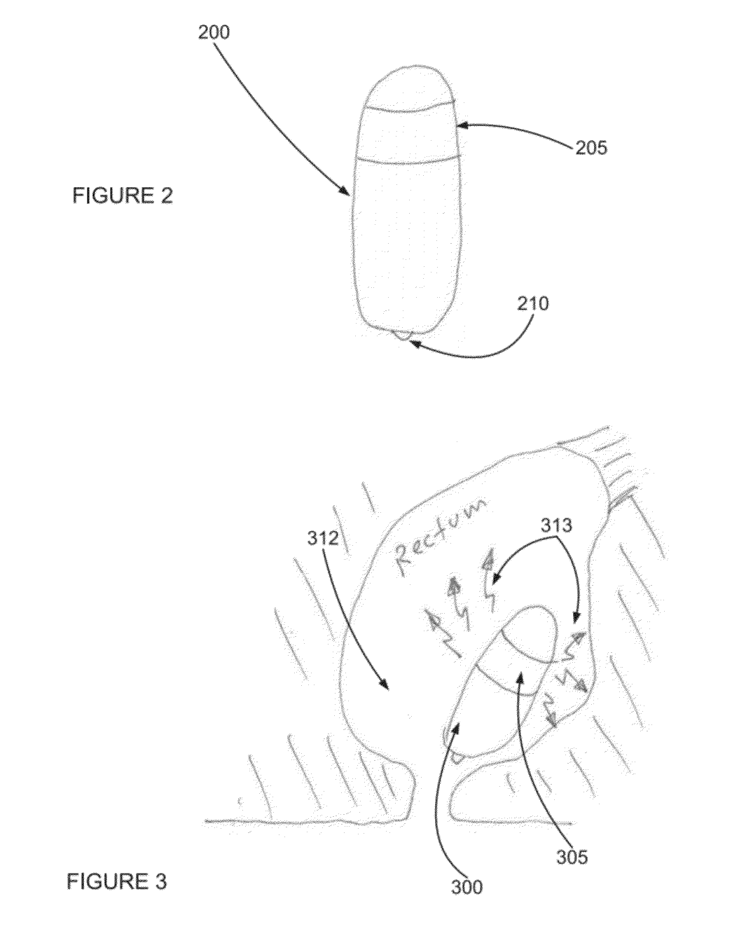

Reference is now made to FIG. 2, which is a simplified drawing of a capsule 200 for providing phototherapeutic illumination according to an example embodiment of the invention.

FIG. 2 depicts the capsule 200 having a window 205 through which the phototherapeutic illumination may be projected. Optionally, the window 205 may extend completely around the circumference of the capsule 200.

In some example embodiments, a phototherapeutic capsule similar to the phototherapeutic capsule described in above-mentioned PCT Patent Application No. PCT IL2011/000257 may be used.

In some embodiments, the phototherapeutic capsule 200 includes a projection 210, or a shaped depression (not shown), which can be used to connect the capsule 200 to an applicator which can optionally serve for inserting the capsule 200 into the rectum and/or colon via the anus.

Inserting a Capsule

Reference is now made to FIG. 3, which is a simplified drawing of a capsule 300 in place within a rectum 312 of a patient, according to an example embodiment of the invention.

FIG. 3 depicts the capsule 300 projecting phototherapeutic illumination 313 via a window 305 toward walls of the rectum 312.

In using an example embodiment for treating a patient, a patient is diagnosed with a disease which is suitable for treatment by phototherapy, and a capsule as described above with reference to FIGS. 2 and 3 is inserted into the rectum.

In some embodiments, a location of a disease and/or lesion is pre-identified, and the location is used to determine how far the treatment capsule/tube should be inserted.

In some embodiments, the capsule is turned on, and/or the phototherapeutic illumination is turned on by removal of the capsule from its package. In some embodiments, the capsule and/or phototherapeutic illumination are turned on by a physician upon removal of the capsule from its package, or at least before insertion into the rectum.

In some embodiments, the capsule is turned on, and/or the phototherapeutic illumination is turned on, by a remote signal, wireless, acoustic, or through a cable.

In some embodiments, a physician inserts the capsule by hand.

In some embodiments, a physician inserts the capsule using a tool, such as an applicator, a basket, or an endoscope, to aid the insertion.

In some embodiments an applicator connected to the capsule is used to insert the capsule into the rectum.

In some embodiments the applicator detached from the capsule when the capsule is in place.

In some embodiments, removal of the applicator from the capsule turns on the phototherapeutic illumination of the capsule.

An Example Embodiment—Including an Applicator

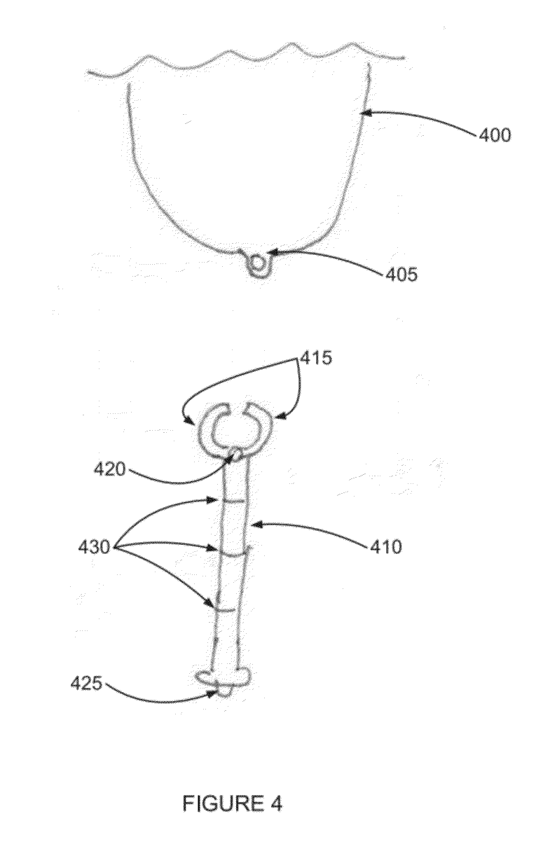

Reference is now made to FIG. 4, which is a simplified drawing of a bottom of a capsule 400 and of an applicator 410 for inserting the capsule into the rectum, according to an example embodiment of the invention.

FIG. 4 depicts the capsule 400 with an optional ring-shaped projection 405, shaped so that the applicator may lock two rotatable arms 415 into the projection 405, thereby clamping the capsule 400 to the applicator 410.

It is noted that persons skilled in the art may devise other methods of clamping the capsule 400 to the applicator 410.

In some embodiments, the applicator 410 has two arms rotatable about a hinge 420. The arms 415 are optionally opened by pushing on a button 425, although other means of opening the arms can be devised by persons skilled in the art.

In some embodiments, the applicator 410 has markings 430 along the body of the applicator 410. The markings 430 correspond to different depth to which the applicator 410 inserts the capsule 400.

In some embodiments the markings 430 have a physiological meaning, such as beginning and end of the rectum.

In some embodiments the markings 430 are of a depth as measured in units of length such as centimeters and/or inches.

The human rectum is the last part of the GI tract and its length is typically 8 to 15 centimeters. Some of the rectum related diseases can be located in any or all of the rectum's length.

In some embodiments the applicator 410 is used to insert the capsule 400 all the way up the rectum.

In some embodiments use of the applicator 410 is optional and may be used especially in those cases where the upper (proximal) or middle (medial) part of the rectum, or the entire rectum are to be treated. For cases where the last part (distal) of the rectum is to be treated—a manual insertion of the capsule 400, without the use of the applicator 410, may be sufficient.

When the capsule 400 is inserted and located in place—a press on the applicator's top side optionally releases a grip of the applicator on the capsule 400 and the applicator 410 may optionally be pulled out without removing the capsule 400 from the rectum.

In some embodiments, once inserted, the capsule 400 is evacuated via the anus through the body's normal stool evacuation mechanisms, after, or even while applying therapeutic light to the rectum wall.

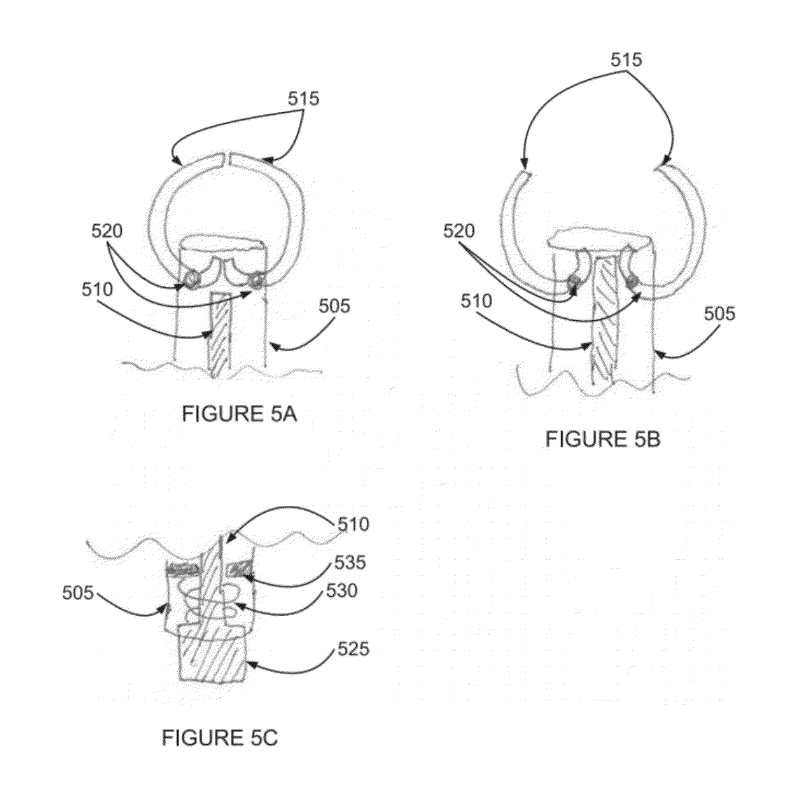

Reference is now made to FIGS. 5A, 5B and 5C, which are more detailed simplified illustrations of technical details of the applicator according to an example embodiment of the invention.

FIG. 5A depicts a tip of a body 505 of the applicator, with arms 515 closed. The arms 515 are optionally kept closed by a spring (not shown). The arms 515 are each rotatably connected to the body 505 by hinges 520. An inner rod 510 is also depicted, which at this time is not pushing the arms 515.

FIG. 5B depicts the tip of the body 505 of the applicator, with the arms 515 being pushed open by the rod 510.

FIG. 5C depicts the other end of the body 505 of the applicator, and a spring 530 which serves to keep the inner rod 510 retracted, and not pushing the arms 515 open, unless the inner rod 510 is pushed against the spring 530.

The spring 530 is depicted as pushing against a flange 535 on the body 505 of the applicator, and a knob 525 on the inner rod 510.

Applying Phototherapy

When the capsule is within the rectum (or colon), the capsule delivers therapeutic light to the rectum using a wavelength and energy which are effective for the disease. The wavelength may be any of the visible wavelength and Near Infra Red (NIR), such as blue, red and NIR, and at energy levels suitable for the wavelength, such as in the range from 0.1 Joule per square centimeter to 2 Joule per square centimeter.

The phototherapeutic illumination is applied using a selected one of several optional delivery protocols: (a) a constant emission of light until an energy source in the capsule is exhausted; (b) a modulated emission of light, where light is turned on for a certain period of time and then turned off for another period of time, in a repetitive manner, possibly until the energy source is exhausted or the body evacuates the capsule from the rectum; and (c) the capsule includes a motion measurement capability which, according to the capsule's speed and/or position, activates therapeutic illumination to provide a specific dose.

The specific dose may be calculated by the illumination intensity, taking into account the speed of movement of the capsule relative to the wall of the rectum, which can optionally be calculated as a duration the illumination is applied to the rectum wall before the capsule moves along and illuminates a different section of the rectum wall.

In some embodiments the speed dependant delivery protocol is optionally based, at least in part, on a movement sensor which can be used within the capsule to detect if the capsule is moving or stationery, and if moving—how fast.

The use of such a sensor provides a potential benefit for effective dose delivery. For example, when the capsule has halted—illumination can be stopped to prevent overdosing the illuminated area. When the capsule moves slowly—the illumination is optionally low (low intensity and/or intermittent illumination) since tissue is exposed longer to the therapeutic illumination. When the capsule is moving rapidly—the illumination intensity is optionally increased, or intermittent illumination is provided with pauses of shorter duration, as tissue exposure time is reduced.

Supplying Power

In some embodiments, the capsule described above with reference to FIGS. 2, 3, and 4 is self-powered, that is, powered by a power supply within the capsule.

In some embodiments the capsule is powered by a power cable leading outside a patient's body to an external battery and/or some other external power supply. In such embodiments, turning the illumination on and off may be performed via the cable, either by controlling the power or by an optional control wire or wires.

In some embodiments the capsule is powered by a power supply included in an applicator, such as the applicator described above with reference to FIGS. 4 and 5A-5C. In such embodiments, turning the illumination on and off may be performed at the applicator, either by controlling the power or by an optional control wire or wires between the capsule and the applicator.

In some embodiments the capsule is optionally initially powered by a power supply external to the capsule, and optionally later powered by a power supply within the capsule.

Un-Tethered, Tethered, and Connected to an Applicator

In some embodiments, the capsule operates un-tethered, that is, separate from a power cable and/or an applicator for all or most of its time of operation. In such embodiments, if optional speed measurement is included, the capsule optionally calculates the therapeutic dose provided to the rectum walls based, at least in part, on the measured speed of movement, and optionally provides an illumination protocol as described above.

In some embodiments, the capsule operates tethered, that is, connected to a power cable. In some such embodiments, if optional speed measurement is included, the capsule optionally calculates the therapeutic dose provided to the rectum walls based, at least in part, on the measured speed of movement, and optionally provides an illumination protocol as described above.

In some such embodiments, if optional speed measurement is included, the capsule optionally calculates the therapeutic dose provided to the rectum walls based, at least in part, on the measured speed of movement, and optionally provides a physician with an indication of when the capsule should be withdrawn from the rectum, or how fast the capsule should be withdrawn from the rectum. In some embodiments, the withdrawing from the rectum is optionally done by pulling on the power cable.

In some such embodiments, if optional speed measurement is included, the capsule optionally measures the speed, and passes data about the speed over the cable to an external control unit. The external control unit optionally calculates the therapeutic dose provided to the rectum walls based, at least in part, on the measured speed of movement, and optionally provides an illumination protocol as described above.

In some such embodiments, if optional speed measurement is included, the capsule optionally measures the speed, and passes data about the speed over the cable to an external control unit. The external control unit optionally calculates the therapeutic dose provided to the rectum walls based, at least in part, on the measured speed of movement, and optionally provides a physician with an indication of when the capsule should be withdrawn from the rectum, or how fast the capsule should be withdrawn from the rectum. In some embodiments, the withdrawing from the rectum is optionally done by pulling on the power cable.

Controlling Distribution of the Intensity of Phototherapeutic Illumination

When providing rectal phototherapy, an energy dose, as measured in Joules per square centimeter, is affected by the diameter of the intestine surrounding the capsule. For example, the distance between the illumination element and the illuminated tissue affects intensity of the illumination. A treated area of the intestine is approximately equal to 2π×(intestine radius)×(length of treated area), assuming a roughly circular intestine.

To obtain an equal dose delivered to the surroundings of the emitting element, the illuminating elements should be placed substantially in the center of the rectum/intestine lumen. During treatment, the capsule, or a rectal tube, is inserted into the rectum, colon, or large intestine, and due to curves and structure of the intestine/rectal lumen, there may not be a constant diameter, or the capsule may not be positioned in the middle of the rectum/intestine in order to deliver an equal dose to the surrounding tissue. The above-described situations may cause a problem—an over-dose or an under-dose of light delivered to the rectum walls, and possibly an unsuccessful therapy.

In some embodiments, the capsule is mechanically centered within the rectum, optionally in a way that does not obstruct the therapeutic illumination.

In some embodiments, an inflatable balloon, for example such as an inflatable balloon used in angioplasty catheters, optionally made of a transparent material such as polyethylene terephthalate (PET) and/or nylon, is located on the capsule and/or on a rectal tube above the illuminating elements. When inflated, the balloon positions the capsule substantially in the middle of the rectum, thus delivering a substantially even dose to the surrounding tissue.

In some embodiments, the balloon is substantially transparent to the wavelengths used for providing phototherapy, such that little energy is lost to passing the phototherapeutic illumination through the balloon.

In some embodiments, a phototherapeutic dosage is corrected to take into account attenuation due to the illumination being provided through the balloon.

A potential advantage of the inflatable balloon is that the balloon may optionally be inflated enough to push and extends the rectum/intestine walls, possibly eliminating some folds in the walls, preventing shading and assuring the walls a more perpendicular direction of illumination.

In some embodiments, for example when using blue wavelength illumination for phototherapy, the illumination penetrates less through tissue than other wavelengths, such as Near Infra-Red. In such cases it is potentially beneficial to eliminate folding of the rectum walls.

In some embodiments, especially when using Near Infra-Red illumination, the illumination penetrates through tissue to a depth which potentially causes less concern about folding of walls.

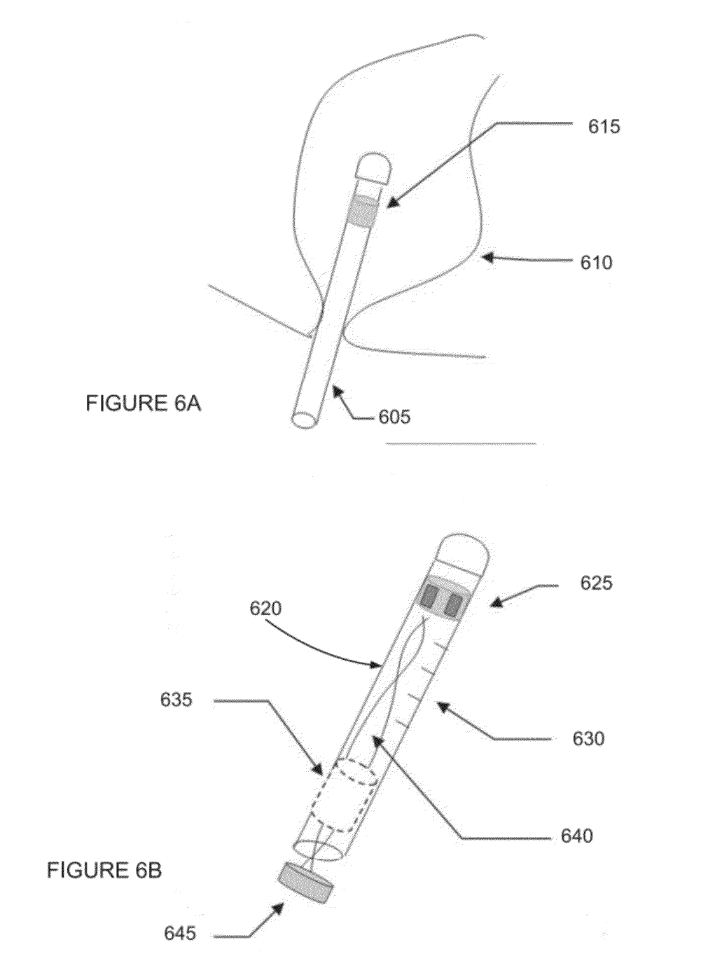

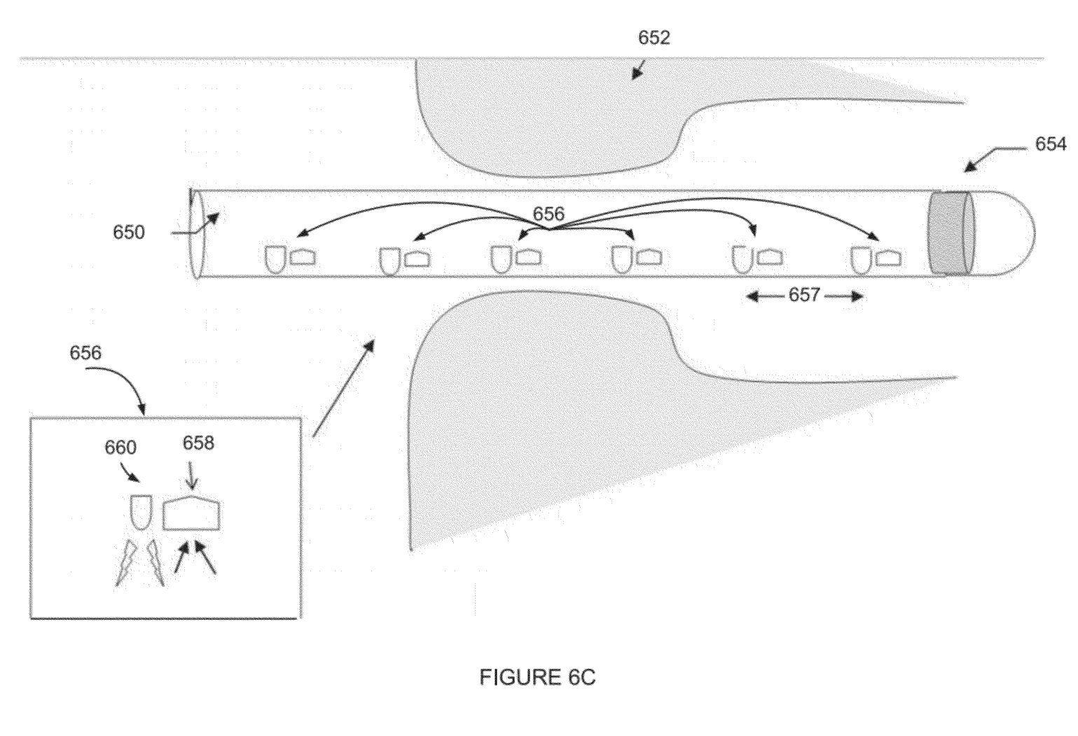

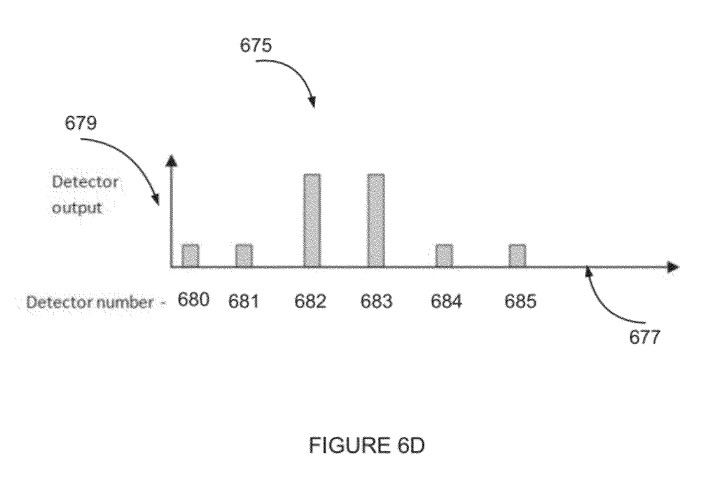

Reference is now made to FIG. 6A, which is a simplified illustration of a therapeutic illumination tube 605 constructed according to an example embodiment of the invention, inserted into a rectum 610.

FIG. 6A depicts the above-described problem.

FIG. 6A depicts the therapeutic illumination tube 605 inserted into the rectum 610. As demonstrated in FIG. 6A, illuminating elements 615 in the therapeutic illumination tube 605 are not centered in the rectum 610, so different energy density is delivered to the rectum 610 walls surrounding the tube 605.

FIG. 6A depicts a therapeutic illumination tube 605 for purpose of depicting an embodiment which is not shaped like a capsule. The tube provides phototherapeutic function similar to the capsule of FIGS. 2, 3, and 4. However, the same problem depicted in FIG. 6A for the therapeutic illumination tube 605 may occur to a capsule and/or to a capsule connected to an applicator. The capsule connected to an applicator behaves in much the same way as the therapeutic illumination tube 605 of FIG. 6A.

Reference is now made to FIG. 6B, which is a simplified illustration of a therapeutic illumination tube 620 constructed according to an example embodiment of the invention.

FIG. 6B depicts the illumination tube 620 having illumination elements 625, and optionally an internal power supply 635, such as a battery, connected by wires 640 to the illumination elements 625.

FIG. 6B is also intended to depict that the internal power supply 635 can be optional in the illumination tube 620, and that an optional connector 645 can be used to connect to and power the illumination elements 625.

FIG. 6B also depicts optional depth markings on the illumination tube 620, which can be used to read out a depth to which the illumination tube 620 has been inserted into the rectum.

In some embodiments of the invention, depth of insertion is read out from an electronic depth measurement component. An example such component is now described.

An Electro-Optic Depth-of-Insertion Measurement Component

Reference is now made to FIG. 6C, which is a simplified illustration of a therapeutic illumination tube 650 having an electro-optical depth measurement component constructed according to an example embodiment of the invention.

FIG. 6C depicts an example embodiment with an electro-optical depth measurement component which includes several light-emitter/light-detector pairs 656 optionally arranged at equal distances 657 along the length of the therapeutic illumination tube 650.

FIG. 6C also depicts illuminating elements 654 optionally arranged around a circumference of the illumination tube 650.

The illumination tube 650 is depicted inserted into a rectum 652.

The light-emitter/light-detector pairs 656 each include a light emitter 660, for example a LED, and a light detector 658, configured to optionally shine light onto the rectum 652 wall, and optionally receive light returned from the rectum 652 wall.

The electro-optic depth-of-insertion measurement component is optionally based on pairs of LEDs and light-detectors located at a distance of 1 centimeter from each other along the tube 650, the LEDs emitting light towards an outer circumference of the tube 650 and the photo-detector detecting light returned from the rectum 652 walls. In a section of the tube 650 located inside the rectum 652 reflections from the rectum 652 walls closer to the tube 650 will produce a bigger response than reflections from the rectum 652 walls further from the tube 650, while a section of the tube which is outside the Rectum will see little or no reflections and produce little or no response.

The electro-optic depth-of-insertion measurement component produces response signals which are optionally interpreted by a control or computing unit to calculate how deep the tube 650 is inserted into the rectum 652. The control unit optionally indicates the depth to a user, and/or provides an indication of a correct or incorrect location, optionally based on a known location for which therapeutic illumination is planned.

Reference is now made to FIG. 6D, which is a simplified graphic illustration 675 of reflection intensity from several light detectors along the therapeutic illumination tube with the electro-optical depth measurement component of FIG. 6C.

An X-axis of the graph 675 represents distance along the illumination tube 650 of FIG. 6C.

A Y-axis of the graph 675 represents intensity of detector output, which is proportional to intensity of reflected light detected the light detectors 658 of FIG. 6C.

Various locations 680 681 682 683 684 685 along the X-axis 677 depict differing heights of reflectance.

Locations 680 and 681 represent very little reflectance, corresponding to locations where the therapeutic illumination tube 650 is outside the rectum.

Locations 682 and 683 represent higher reflectance, corresponding to locations where the therapeutic illumination tube 650 is close to the anus walls.

Locations 684 and 685 represent lower reflectance compared to the anus locations, corresponding to locations where the therapeutic illumination tube 650 is inside the rectum, yet relatively distant from the walls of the rectum.

The above simplified illustration depicts how a location of the anus may be detected. Since distance between the detectors is known, a depth of insertion of the therapeutic illumination tube 650 relative to the anus is optionally computed.

The optional electro-optical depth measurement component is used to measure the location, or depth of insertion, of the tube within the rectum.

In some embodiments, the above measurement is optionally used by a patient self-administering therapeutic illumination, since the patient typically finds it hard to see a tube's distance markings and so cannot place the tube in the rectum, and optionally pull the tube out the rectum, in a manner required by a therapeutic illumination treatment protocol which may specify depths and duration of illumination and/or speed of withdrawal from or insertion into the rectum.

In some embodiments the above measurement is used by a care giver who needs to provide accurate treatment according to the therapeutic illumination treatment protocol.

Centering the Phototherapeutic Device with a Lumen

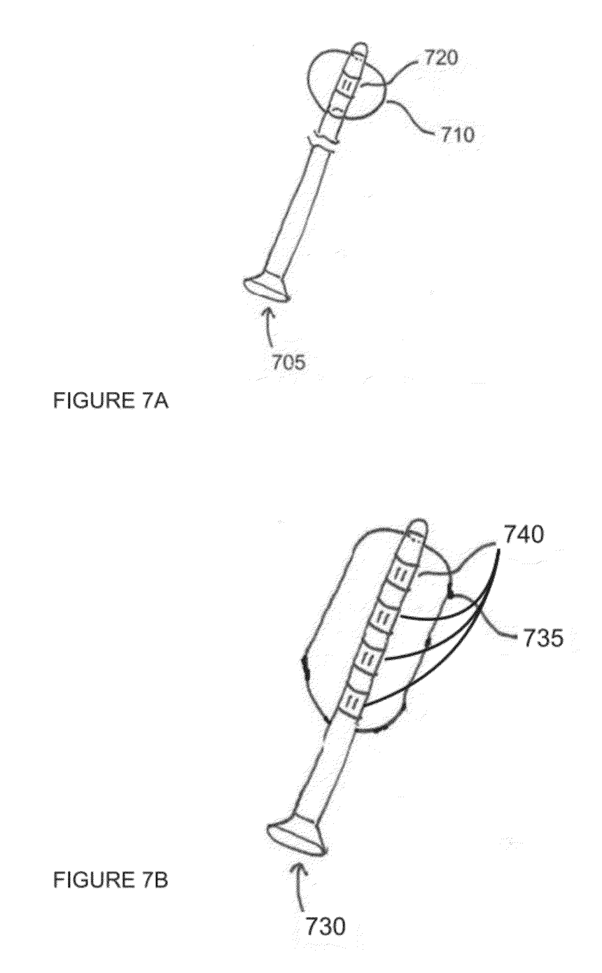

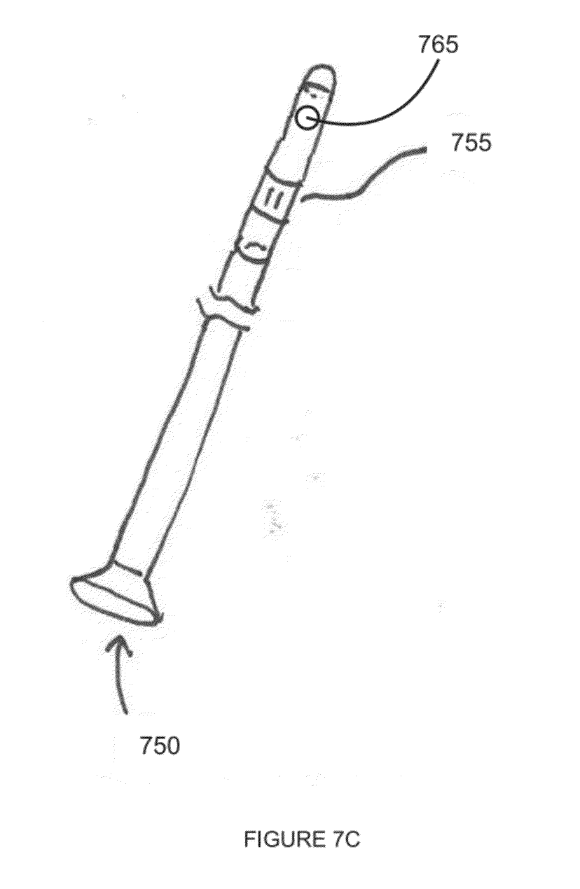

Reference is now made to FIG. 7A, which is a simplified illustration of a therapeutic illumination tube 705 with an inflatable balloon 710 according to an example embodiment of the invention.

FIG. 7A depicts the therapeutic illumination tube 705 with the inflatable balloon 710.

In some embodiments, an open end of the therapeutic illumination tube 705 is used to optionally flow air into the therapeutic illumination tube 705, and optionally inflate the inflatable balloon 710 located around phototherapeutic illuminating elements 720.

Reference is now made to FIG. 7B, which is a simplified illustration of a therapeutic illumination tube 730 with an inflatable balloon 735 according to another example embodiment of the invention.

FIG. 7B depicts the therapeutic illumination tube 730 with the inflatable balloon 735 having phototherapeutic illuminating elements 740 along all a substantial part of its length, and the inflatable balloon 735 located around the phototherapeutic illuminating elements 740.

In some embodiments, the phototherapeutic illuminating elements 740 are separate light sources, such as separate LEDs. In some embodiments one light source, or a few light sources, are used for illumination, light from one light source being guided to project along a length of the therapeutic illumination tube 730. In some embodiments one light source, or a few light sources, are used for illumination, light from one light source being guided to project along a length of the therapeutic illumination tube 730, in a manner termed a “leaky light guide”, with light being guided along the tube and projecting out of the tube along its path.

In some embodiments, the phototherapeutic illumination is provided by a light pipe (not shown) guiding therapeutic illumination through the therapeutic illumination tube 730, and exiting to illuminate the rectum walls along a length of the therapeutic illumination tube 730, and/or at specific locations, similar to the locations of the phototherapeutic illuminating elements 740.

In some embodiments, illumination intensity exiting from the light pipe (not shown) is optionally adjusted by a device, such as, by way of a non-limiting example, a liquid crystal adjusting transferred intensity, so that the illumination along the walls of the rectum is substantially even.

A Capsule or Illuminating Tube with a Camera

Reference is now made to FIG. 7C, which is a simplified illustration of a therapeutic illumination tube 750 with a camera 765 according to an example embodiment of the invention.

FIG. 7C depicts the therapeutic illumination tube 750 with the camera 765 and one or more phototherapeutic illuminating elements 755.

In some embodiments the camera 765 is configured to view to a side of the tube, located adjacent to the phototherapeutic illuminating elements 755, so that the camera 765 views the same area as the phototherapeutic illuminating elements 755 illuminate.

In some embodiments, a physician optionally activates therapeutic illumination when the physician views a diseased area.

In some embodiments, a physician optionally scans the rectum discovering diseased areas, and then activates therapeutic illumination at the diseased areas.

In some embodiments, a physician optionally scans the rectum discovering diseased areas, and optionally records images and locations of the diseased areas. The locations are optionally recorded using polar coordinates which include depth-of-insertion of the tube 750 (r-coordinate), and orientation of the tube 750 (θ-coordinate as measured from a reference).

In some embodiments, therapeutic illumination is optionally automatically provided based on the r and θ coordinates which were recorded for the diseased area(s).

In some embodiments, the therapeutic illumination is optionally automatically provided based on the tube 750 being moved along the rectum by the physician, a therapeutic dose being provided at the recorded locations, and instruction to move the tube 750 along being provided to a physician or a caregiver.

In some embodiments the camera 765 is configured to view to a side of the tube, in a same direction as the phototherapeutic illuminating elements 755 project the illumination, but not necessarily viewing the same area as the phototherapeutic illuminating elements 755 illuminate. In such embodiments either one of, or both of, the scanning and the recording described above are optionally performed, and the location-dependent illumination described above is optionally performed.

In some embodiments (not shown) the camera 765 is configured to look forward, along the long axis of the tube, further along the GI tract relative to where the phototherapeutic illuminating elements 755 project the illumination. In such embodiments either one of, or both of, the scanning and the recording described above are optionally performed, and the location-dependent illumination described above is optionally performed.

In some embodiments (not shown), the therapeutic illumination is provided out of the inner end of the illumination tube. The therapeutic illumination is optionally provided as a cone of light, or as a hollow cone of light.

In some embodiments, the illumination intensity is optionally adjusted, optionally by adjusting which illuminating elements are turned on, and/or by a passive device such as a Fresnel lens, and/or by an active device such as by a liquid crystal adjusting transferred intensity, so that the illumination along the walls of the rectum is substantially even, that is a substantially equal intensity per unit area.

In some embodiments, the camera 765 is used to aid a physician in pushing the capsule or illumination tube from the rectum to the sigmoid colon, which involves negotiating a sharp turn.

A Centering Balloon Combined with a Camera

In some embodiments, a centering device, such as the centering balloon 710 of FIG. 7A, is combined with a camera.

In some embodiments, the camera is placed outside of an area covered by the balloon, such as, by way of a non-limiting example, the arrangement depicted in FIG. 7C, in which a balloon may be placed over the phototherapeutic illuminating elements 755, while the camera 765 of FIG. 7C is outside the balloon.

In some such embodiments, the camera may be configured to look forward, further along the GI tract relative to where the phototherapeutic illuminating elements 755 project the illumination, also outside coverage of the balloon.

In some embodiments, the balloon is made substantially transparent to wavelengths employed by the camera for imaging, optionally providing a clear enough image for detection of diseased areas.

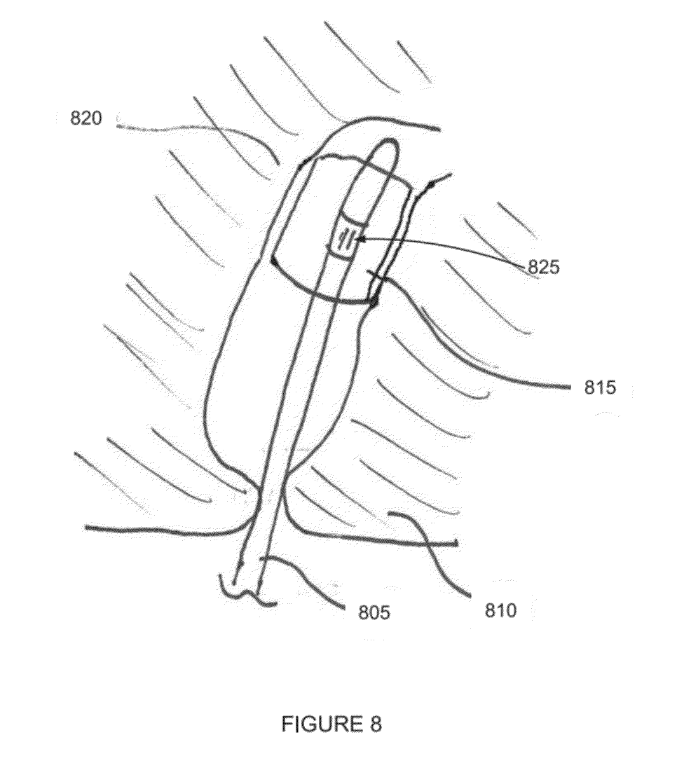

Reference is now made to FIG. 8, which is a simplified illustration of a therapeutic illumination tube 805 with an inflatable balloon 815 inserted into a rectum 810 according to an example embodiment of the invention.

FIG. 8 depicts a position of the therapeutic illumination tube 805 in the rectum 810 after the inflatable balloon 815 was inflated. As depicted in the drawing, illuminating elements 825 are now positioned in the center of the rectum 810 with an equal distance from rectum walls 820, which potentially enables a more even distribution of therapeutic illumination to the rectum walls 820.

In some embodiments, when the balloon 815 inflates within a rectum, pressing walls of the rectum, the balloon 815 reaches an elongated cylindrical shape which conforms to the inside of the rectum, more like the cylindrical shape of the balloon 735 of FIG. 7B than the round shape of the balloon 710 of FIG. 7A.

A simplified example application of an embodiment of the invention for providing phototherapy to the rectum is now provided.

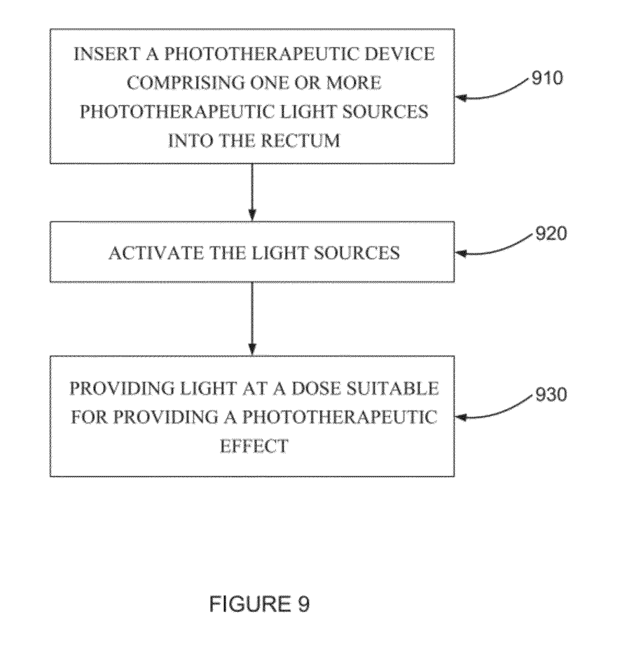

Reference is now made to FIG. 9, which is a simplified flow chart illustration of a method for providing phototherapy to the rectum according to an example embodiment of the invention.

The method of FIG. 9 includes:

inserting a phototherapeutic device comprising one or more phototherapeutic light sources into the rectum (910);

activating the light sources (920); and

providing light at a dose suitable for providing a phototherapeutic effect (930).

A simplified example application of an embodiment of the invention for providing phototherapy to the rectum, using both a camera and phototherapeutic illumination is now provided.

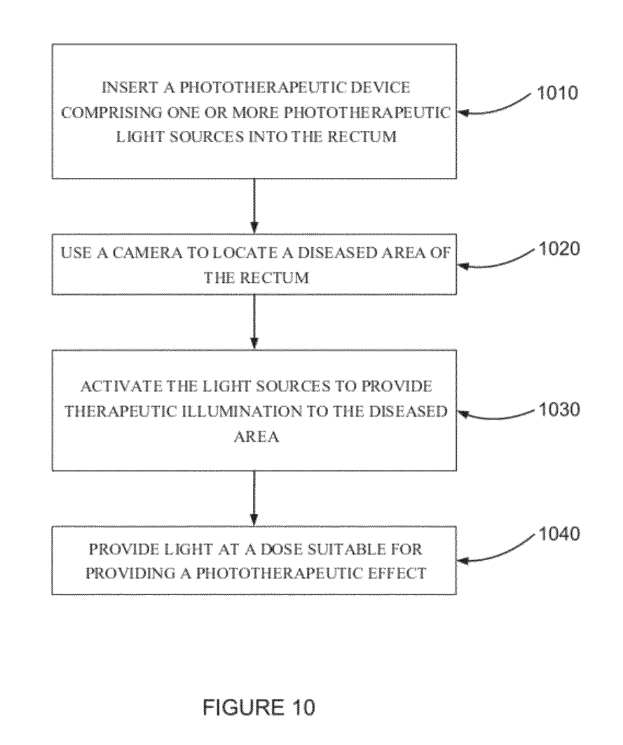

Reference is now made to FIG. 10, which is a simplified flow chart illustration of a method for providing phototherapy to the rectum according to an example embodiment of the invention.

The method of FIG. 10 includes:

inserting a phototherapeutic device comprising one or more phototherapeutic light sources into the rectum (1010);

using a camera to locate a diseased area of the rectum (1020)

activating the light sources to provide therapeutic illumination to the diseased area (1030); and

providing light at a dose suitable for providing a phototherapeutic effect (1040).

In some embodiments, a different therapeutic illumination dose is provided to a portion of the rectum which has mucus-lined walls than to a portion of the rectum which has non-mucus-lined walls.

It is expected that during the life of a patent maturing from this application many relevant therapeutic illuminating elements and inflatable balloons will be developed and the scope of the terms therapeutic illuminating elements and inflatable balloons is intended to include all such new technologies a priori.

The terms “comprising”, “including”, “having” and their conjugates mean “including but not limited to”.

The term “consisting of” is intended to mean “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a unit” or “at least one unit” may include a plurality of units, including combinations thereof.

The words “example” and “exemplary” are used herein to mean “serving as an example, instance or illustration”. Any embodiment described as an “example or “exemplary” is not necessarily to be construed as preferred or advantageous over other embodiments and/or to exclude the incorporation of features from other embodiments.

The word “optionally” is used herein to mean “is provided in some embodiments and not provided in other embodiments”. Any particular embodiment of the invention may include a plurality of “optional” features unless such features conflict.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

As used herein, the term “treating” includes abrogating, substantially inhibiting, slowing or reversing the progression of a condition, substantially ameliorating clinical or aesthetical symptoms of a condition or substantially preventing the appearance of clinical or aesthetical symptoms of a condition.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination or as suitable in any other described embodiment of the invention. Certain features described in the context of various embodiments are not to be considered essential features of those embodiments, unless the embodiment is inoperative without those elements.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting.

What is claimed is:

1. A phototherapeutic system for providing phototherapy to a patient's lower parts of a gastrointestinal (GI) tract, the phototherapeutic system comprising a capsule sized and shaped for insertion into the rectum, the capsule comprising one or more phototherapeutic light sources.

2. The system of claim 1 in which the capsule is longer than 4 centimeters.

3. The system of claim 1 in which the capsule diameter is more than 2 centimeters.

4. The system of claim 1 and further comprising a centering component configured to center the capsule relative to a diameter of the rectum.

5. The system of claim 4 in which the centering component is configured to exert pressure on walls of the rectum, thereby flattening folds in the walls.

6. The system of claim 4 in which the centering component is an inflatable balloon surrounding the capsule.

7. The system of claim 4 in which the centering component is substantially transparent to a wavelength used for the providing phototherapy.

8. The system of claim 1 in which the phototherapeutic light sources comprises a plurality of phototherapeutic light sources, and the phototherapeutic light sources are distributed along a length of the capsule.

9. The system of claim 4 in which:
the phototherapeutic light sources comprise a plurality of phototherapeutic light sources;
the phototherapeutic light sources are distributed along a length of the capsule; and
the centering component surrounds the phototherapeutic light sources distributed along the length of the capsule.

10. The system of claim 1 in which the capsule further comprises:
a movement determination unit for calculating speed of movement of the capsule in the rectum.

11. The system of claim 10 in which the capsule further comprises a controller unit for activating one or more of the one or more light sources for delivering a therapeutic illumination dose to a target site in the GI tract, based, at least in part, on a determined speed calculated based, at least in part, on movement determined by the movement determination unit.

12. The system of claim 10 and further comprising a controller unit, external to the capsule, for activating one or more of the one or more light sources for delivering a therapeutic illumination dose to a target site in the GI tract, based, at least in part, on a determined speed calculated based, at least in part, on movement determined by the movement determination unit.

13. The system of claim 12 in which the controller unit is comprised in an applicator connected to the capsule.

14. The system of claim 12 in which the controller unit is comprised in a computer configured to be external to a patient's body.

15. The system of claim 11 and further comprising a depth-of-insertion measurement component.

16. The system of claim 15 in which the depth-of-insertion measurement component is an electronic depth-of-insertion measurement component connected to the controller unit.

17. The system of claim 11 in which the controller unit is configured to indicate to a physician when a therapeutic dose has been completely administered to a location in the rectum, based, at least in part, on the determined movement and on a required dose.

18. The system of claim 12 in which the controller unit is configured to indicate to a physician an indication for changing a speed of withdrawal of the capsule from a patient's rectum based, at least in part, on the determined movement and on a required dose.

19. The system of claim 1 and further comprising an applicator for inserting the capsule into the rectum, wherein the applicator is shaped for manipulating from outside a patient's body.

20. The system of claim 19 in which the applicator comprises a mechanical connector for connecting to the capsule, and wherein the applicator is configured to enable disconnection from the capsule when the capsule is within a patient's rectum.

21. The system of claim 1 and further comprising a power supply built within the capsule.

22. The system of claim 1 and further comprising a power cable configured for connecting the capsule to a power supply outside a patient's body.

23. A method for intra-luminal phototherapy of the lower parts of the gastrointestinal (GI) tract comprising:
inserting a phototherapeutic device comprising one or more phototherapeutic light sources into the rectum;
activating the light sources; and
providing light at a dose suitable for providing a phototherapeutic effect.

24. The method of claim 23 and further comprising substantially centering the phototherapeutic device relative to a circumference of the rectum.

25. The method of claim 24 in which the centering exerts pressure on walls of the rectum, thereby flattening folds in the walls.

26. The method of claim 24 in which the centering comprises inflating a balloon surrounding the phototherapeutic device.

27. The method of claim 23 and further comprising measuring a depth-of-insertion of the phototherapeutic device in the rectum by an electronic depth-of-insertion measurement component, and providing the light based, at least in part, on the measuring.

28. The method of claim 23 in which the phototherapeutic device comprises a capsule connected to an applicator, and further comprising detaching the applicator and removing the applicator from the rectum, leaving the capsule inside the rectum.

29. The method of claim 23 and further comprising:
measuring movement of the phototherapeutic device relative to the rectum, and using the movement to calculate the dose of therapeutic lighting received by walls of the rectum, wherein the measurement is used to indicate to a user at what rate the phototherapeutic device should be pulled out of the rectum while therapeutic light is being provided.

30. The method of claim 23 in which the phototherapeutic device further comprises a camera, and the method for intra-luminal phototherapy further comprises using the camera to locate a diseased area of the rectum.

31. The method of claim 30 and further comprising recording images of the diseased area.
